(19) United States
(12) Patent Application Publication
(10) Pub. No.: US 2017/0112382 A1
(43) Pub. Date: Apr. 27, 2017

(54) PULSE-WAVE DETECTION METHOD, PULSE-WAVE DETECTION DEVICE, AND COMPUTER-READABLE RECORDING MEDIUM

(71) Applicant: FUJITSU LIMITED, Kawasaki-shi (JP)

(72) Inventors: YASUYUKI NAKATA, Zama (JP); Akihiro Inomata, Atsugi (JP); Takuro Oya, Kawasaki (JP); Masato Sakata, Isehara (JP)

(73) Assignee: FUJITSU LIMITED, Kawasaki (JP)

(21) Appl. No.: 15/397,000

(22) Filed: Jan. 3, 2017

Related U.S. Application Data
(63) Continuation of application No. PCT/JP2014/068094, filed on Jul. 7, 2014.

(51) Int. Cl. A61B 5/00; A61B 5/1171; A61B 5/024

(52) U.S. Cl. CPC A61B 5/0077; A61B 5/024; A61B 5/1176; A61B 5/0022

(57) ABSTRACT

A pulse-wave detection device acquires an image. Furthermore, the pulse-wave detection device executes face detection on the image. Furthermore, the pulse-wave detection device sets the identical region of interest in the frame, of which the image is acquired, and the previous frame to the frame in accordance with a result of the face detection. Moreover, the pulse-wave detection device detects a pulse wave signal based on a difference in brightness obtained between the frame and the previous frame.

### CROSS-REFERENCE TO RELATED APPLICATION

This application is a continuation of International Application No. PCT/JP2014/068094, filed on Jul. 7, 2014, the entire contents of which are incorporated herein by reference.

### FIELD

The embodiments discussed herein are related to a pulse-wave detection method, a pulse-wave detection program, and a pulse-wave detection device.

### BACKGROUND

As an example of the technology for detecting fluctuation in the volume of blood, what is called a pulse wave, there is a disclosed heartbeat measurement method for measuring heartbeats from images that are taken by users. According to the heartbeat measurement method, the face region is detected from the image captured by a Web camera, and the average brightness value in the face region is calculated for each RGB component. Furthermore, in the heartbeat measurement method, Independent Component Analysis (ICA) is applied to the time-series data on the average brightness value for each RGB, and then Fast Fourier Transform (FFT) is applied to one of the three component waveforms on which the ICA has been performed. In addition, according to the heartbeat measurement method, the number of heartbeats is estimated based on the peak frequency that is obtained by the FFT.

[Patent document 1] Japanese Laid-open Patent Publication No. 2003-331268

However, with the above-described technology, the accuracy with which pulse waves are detected is sometimes decreased as described below.

Specifically, if the number of heartbeats is measured from an image, the area of the living body, where a change in the brightness occurs due to pulse waves, is extracted as the region of interest; therefore, face detection using template matching, or the like, is executed on the image captured by the Web camera. However, during face detection, there occurs an error in the position where the face region is detected and furthermore, even if the face does not move on the image, the face region is not always detected on the same position of the image. Therefore, even if the face does not move, the position where the face region is detected is sometimes varied in frames of the image. In this case, in time-series data on the average brightness value that is acquired from images, changes in the brightness due to variations in the position where the face region is detected appear more largely than changes in the brightness due to pulse waves and, as a result, the accuracy with which pulse waves are detected is decreased.

### SUMMARY

According to an aspect of an embodiment, a pulse-wave detection method includes: acquiring, by a processor, an image; executing, by the processor, face detection on the image; setting, by the processor, an identical region of interest in a frame, of which the image is acquired, and a previous frame to the frame in accordance with a result of the face detection; and detecting, by the processor, a pulse wave signal based on a difference in brightness obtained between the frame and the previous frame.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram that illustrates a functional configuration of a pulse-wave detection device according to a first embodiment;

FIG. 2 is a diagram that illustrates an example of calculation of the arrangement position of the ROI;

FIG. 3 is a flowchart that illustrates the steps of a pulse-wave detection process according to the first embodiment;

FIG. 4 is a graph that illustrates an example of the relationship between a change in the position of the ROI and a change in the brightness;

FIG. 5 is a graph that illustrates an example of the relationship between a change in the position of the ROI and a change in the brightness;

FIG. 6 is a graph that illustrates an example of changes in the brightness due to changes in the position of the face;

FIG. 7 is a graph that illustrates an example of the change in the brightness due to pulses;

FIG. 8 is a graph that illustrates an example of time changes in the brightness;

FIG. 9 is a block diagram that illustrates a functional configuration of a pulse-wave detection device according to a second embodiment;

FIG. 10 is a diagram that illustrates an example of a weighting method;

FIG. 11 is a diagram that illustrates an example of the weighting method;

FIG. 12 is a flowchart that illustrates the steps of a pulse-wave detection process according to the second embodiment;

FIG. 13 is a block diagram that illustrates a functional configuration of a pulse-wave detection device according to a third embodiment;

FIG. 14 is a diagram that illustrates an example of shift of the ROI;

FIG. 15 is a diagram that illustrates an example of extraction of a block;

FIG. 16 is a flowchart that illustrates the steps of a pulse-wave detection process according to a third embodiment; and

FIG. 17 is a diagram that illustrates an example of the computer that executes the pulse-wave detection program according to the first embodiment to a fourth embodiment.

### DESCRIPTION OF EMBODIMENTS

Preferred embodiments will be explained with reference to accompanying drawings. Furthermore, embodiments do not limit the disclosed technology. Moreover, embodiments may be combined as appropriate to the extent that there is no contradiction of processing details.

### [a] First Embodiment

### Configuration of the Pulse-Wave Detection Device

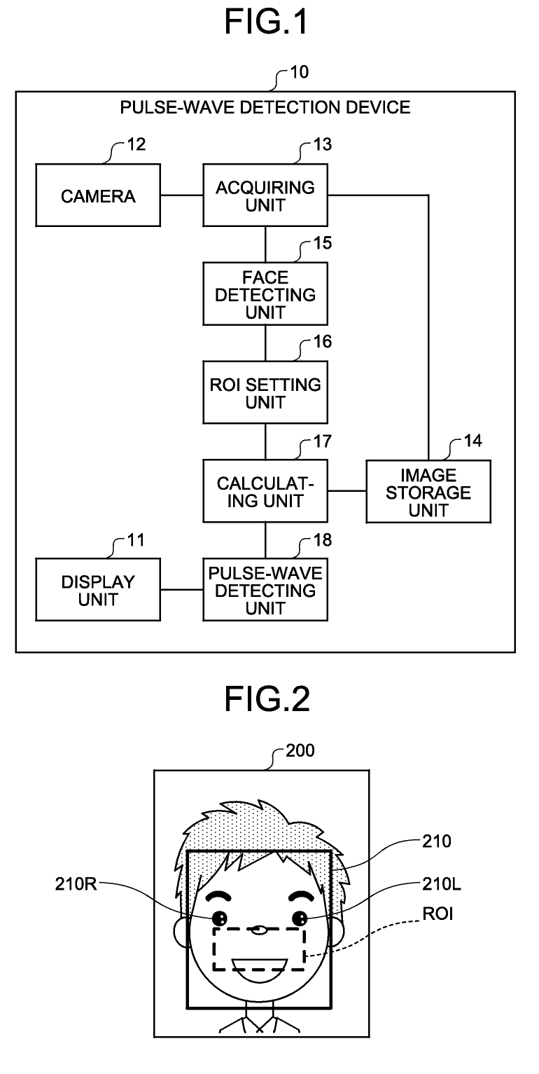

FIG. 1 is a block diagram that illustrates a functional configuration of a pulse-wave detection device according to a first embodiment. A pulse-wave detection device 10, illustrated in FIG. 1, performs a pulse-wave detection process to measure pulse waves, i.e., fluctuation in the volume of blood due to heart strokes, by using images that capture the living body under general environmental light, such as sunlight or room light, without bringing a measurement device into contact with the human body.

According to an embodiment, the pulse-wave detection device 10 may be implemented when the pulse-wave detection program, which provides the above-described pulse-wave detection process as package software or online software, is installed in a desired computer. For example, the above-described pulse-wave detection program is installed in the overall mobile terminal devices including digital cameras, tablet terminals, or slate terminals, as well as mobile communication terminals, such as smartphones, mobile phones, or Personal Handy-phone System (PHS). Thus, the mobile terminal device may function as the pulse-wave detection device 10. Furthermore, although the pulse-wave detection device 10 is here implemented as a mobile terminal device in the illustrated case, stationary terminal devices, such as personal computers, may be implemented as the pulse-wave detection device 10.

As illustrated in FIG. 1, the pulse-wave detection device 10 includes a display unit 11, a camera 12, an acquiring unit 13, an image storage unit 14, a face detecting unit 15, an ROI (Region of Interest) setting unit 16, a calculating unit 17, and a pulse-wave detecting unit 18.

The display unit 11 is a display device that displays various types of information.

According to an embodiment, the display unit 11 may use a monitor or a display, or it may be also integrated with an input device so that it is implemented as a touch panel. For example, the display unit 11 displays images output from the operating system (OS) or application programs, operated in the pulse-wave detection device 10, or images fed from external devices.

The camera 12 is an image taking device that includes an imaging element, such as a charge-coupled device (CCD) or a complementary metal-oxide semiconductor (CMOS).

According to an embodiment, an in-camera or an out-camera provided in the mobile terminal device as standard features may be also used as the camera 12. According to another embodiment, the camera 12 may be also implemented by connecting a Web camera or a digital camera via an external terminal. Here, in the illustrated example, the pulse-wave detection device 10 includes the camera 12; however, if images may be acquired via networks or storage devices including storage media, the pulse-wave detection device 10 does not always need to include the camera 12.

For example, the camera 12 is capable of capturing rectangular images with 320 pixels×240 pixels in horizontal and vertical. For example, in the case of gray scale, each pixel is given as the tone value (brightness) of lightness. For example, the tone value of the brightness (L) of the pixel at the coordinates (i, j), represented by using integers i, j, is given by using the digital value L(i, j) in 8 bits, or the like. Furthermore, in the case of color images, each pixel is given as the tone value of the red (R) component, the green (G) component, and the blue (B) component. For example, the tone value in R, G, and B of the pixel at the coordinates (i, j), represented by using the integers i, j, is given by using the digital values R(i, j), G(i, j), and B(i, j), or the like. Furthermore, other color systems, such as the Hue Saturation Value (HSV) color system or the YUV color system, which are obtained by converting the combination of RGB or RGB values, may be used.

Here, an explanation is given of an example of the situation where images, used for detection of pulse waves, are captured. For example, in the assumed case, the pulse-wave detection device 10 is implemented as a mobile terminal device, and the in-camera, included in the mobile terminal device, takes images of the user's face. Generally, the in-camera is provided on the same side as the side where the screen of the display unit 11 is present. Therefore, if the user views images displayed on the display unit 11, the user's face is opposed to the screen of the display unit 11. In this way, if the user views images displayed on the screen, the user's face is opposed to not only the display unit 11 but also the camera 12 provided on the same side as the display unit 11.

If image capturing is executed under the above-described condition, images captured by the camera 12 have for example the following tendency. For example, there is a tendency that the user's face is likely to appear on the image captured by the camera 12. Furthermore, it is often the case that, if the user's face appears on the image, the user's face is likely to be frontally opposed to the screen. In addition, there is a tendency that many images are acquired by being taken at the same distance from the screen. Therefore, it is expected that the size of the user's face, which appears on the image, is the same in frames or is changed to such a degree that it is regarded as being the same. Hence, if the region of interest, what is called ROI, which is used for detection of pulse waves, is set in the face region detected from images, the size of the ROI may be the same, although if not the position of the ROI set on the image.

Furthermore, the condition for executing the above-described pulse-wave detection program on the processor of the pulse-wave detection device 10 may include the following conditions. For example, it may be started up when a start-up operation is performed via an undepicted input device, or it may be also started up in the background when contents are displayed on the display unit 11.

For example, if the above-described pulse-wave detection program is executed in the background, the camera 12 starts to capture images in the background while contents are displayed on the display unit 11. Thus, the state of the user viewing the contents with the face opposing to the screen of the display unit 11 is captured as an image. The contents may be any type of displayed materials, including documents, videos, or moving images, and they may be stored in the pulse-wave detection device 10 or may be acquired from external devices, such as Web servers. As described above, after contents are displayed, there is a high possibility that the user watches the display unit 11 until viewing of the contents is terminated; therefore, it is expected that images where the user's face appears, i.e., images applicable to detection of pulse waves, are continuously acquired. Furthermore, if pulse waves are detectable from images captured by the camera 12 in the background while contents are displayed on the display unit 11, health management may be executed or evaluation on contents including still images or moving images may be executed without making the user of the pulse-wave detection device 10 aware of it.

Furthermore, if the above-described pulse-wave detection program is started up due to a start-up operation of the user, the guidance for the capturing procedure may be provided through image display by the display unit 11, sound output by an undepicted speaker, or the like. For example, if the pulse-wave detection program is started up via an input device, it activates the camera 12. Accordingly, the camera 12 starts to capture an image of the object that is included in the capturing range of the camera 12. Here, the pulse-wave detection program is capable of displaying images, captured by the camera 12, on the display unit 11 and also displaying the target position, in which the user's nose appears, as the target on the image displayed on the display unit 11. Thus, image capturing may be executed in such a manner that the user's nose among the facial parts, such as eye, ear, nose, or mouth, falls into the central part of the capturing range.

The acquiring unit 13 is a processing unit that acquires images.

According to an embodiment, the acquiring unit 13 acquires images captured by the camera 12. According to another embodiment, the acquiring unit 13 may also acquire images via auxiliary storage devices, such as hard disk drive (HDD), solid state drive (SSD), or optical disk, or removable media, such as memory card or Universal Serial Bus (USB) memory. According to further another embodiment, the acquiring unit 13 may also acquire images by receiving them from external devices via a network. Here, in the illustrated example, the acquiring unit 13 performs processing by using image data, such as two-dimensional bitmap data or vector data, obtained from output of imaging elements, such as CCD or CMOS; however, it is also possible that signals, output from the single detector, are directly acquired and the subsequent processing is performed.

The image storage unit 14 is a storage unit that stores images.

According to an embodiment, the image storage unit 14 stores images acquired during capturing each time the capturing is executed by the camera 12. Here, the image storage unit 14 may store moving images that are encoded by using a predetermined compression coding method, or it may store a set of still images where the user's face appears. Furthermore, the image storage unit 14 does not always need to store images permanently. For example, if a predetermined time has elapsed after an image is registered, the image may be deleted from the image storage unit 14. Furthermore, it is also possible that images from the latest frame, registered in the image storage unit 14, to the predetermined previous frames are stored in the image storage unit 14 while the frames registered before them are deleted from the image storage unit 14. Here, in the illustrated example, images captured by the camera 12 are stored; however, images received via a network may be stored.

The face detecting unit 15 is a processing unit that executes face detection on images acquired by the acquiring unit 13.

According to an embodiment, the face detecting unit 15 executes face recognition, such as template matching, on images, thereby recognizing facial organs, what are called facial parts, such as eyes, ears, nose, or mouth. Furthermore, the face detecting unit 15 extracts, as the face region, the region in a predetermined range, including facial parts, e.g., eyes, nose, and mouth, from the image acquired by the acquiring unit 13. Then, the face detecting unit 15 outputs the position of the face region on the image to the subsequent processing unit, that is, the ROI setting unit 16. For example, if the shape of the region, extracted as the face region, is rectangular, the face detecting unit 15 may output the coordinates of the four vertices that form the face region to the ROI setting unit 16. Here, the face detecting unit 15 may also output, to the ROI setting unit 16, the coordinates of any one of the vertex among the four vertices that form the face region and the height and the width of the face region. Furthermore, the face detecting unit 15 may also output the position of the facial part included in the image instead of the face region.

The ROI setting unit 16 is a processing unit that sets the ROI.

According to an embodiment, the ROI setting unit 16 sets the same ROI in successive frames each time an image is acquired by the acquiring unit 13. For example, if the Nth frame is acquired by the acquiring unit 13, the ROI setting unit 16 calculates the arrangement positions of the ROIs that are set in the Nth frame and the N−1th frame by using the image corresponding to the Nth frame as a reference. The arrangement position of the ROI may be calculated from, for example, the face detection result of the image that corresponds to the Nth frame. Furthermore, if a rectangle is used as the shape of the ROI, the arrangement position of the ROI may be represented by using, for example, the coordinates of any of the vertices of the rectangle or the coordinates of the center of gravity. Furthermore, in the case described below, for example, the size of the ROI is fixed; however, it is obvious that the size of the ROI may be enlarged or reduced in accordance with a face detection result. Furthermore, the Nth frame is sometimes described as “frame N” below. In addition, frames in other numbers, e.g., the N−1th frame, are sometimes described according to the description of the Nth frame.

Specifically, the ROI setting unit 16 calculates, as the arrangement position of the ROI, the position that is vertically downward from the eyes included in the face region. FIG. 2 is a diagram that illustrates an example of calculation of the arrangement position of the ROI. The reference numeral 200, illustrated in FIG. 2, denotes the image acquired by the acquiring unit 13, and the reference numeral 210 denotes the face region that is detected as a face from the image 200. As illustrated in FIG. 2, as the arrangement position of the ROI is calculated, for example, the position that is vertically downward from a left eye 210L and a right eye 210R included in the face region 210. The reason why the position vertically downward from the eyes 210L and 210R is the arrangement position of the ROI as described above is to prevent brightness changes due to blinks of the eyes included in the ROI from occurring in pulse wave signals. Furthermore, the reason why the width of the ROI in a horizontal direction is nearly equal to the width of the eyes 210L and 210R is that there is a high possibility that the ROI has a high brightness gradient due to a major difference in the reflection direction of illumination, or the like, which is caused by a large change in the outline of the face outside the eyes compared to that inside the eyes. As described above, after the arrangement position of the ROI is calculated, the ROI setting unit 16 sets the same ROI in the previously calculated arrangement positions with regard to the image in the frame N and the image in the frame N−1.

The calculating unit 17 is a processing unit that calculates a difference in the brightness of the ROI in frames of an image.

According to an embodiment, for each frame from the frame N and the frame N−1, the calculating unit 17 calculates the representative value of the brightness in the ROI that is set in the frame. Here, if the representative value of the brightness in the ROI is obtained with regard to the previously acquired frame N−1, the image in the frame N−1 stored in the image storage unit 14 may be used. If the representative value of the brightness is obtained in this manner, for example, the brightness value of the G component, which has higher light absorption characteristics of hemoglobin among the RGB components, is used. For example, the calculating unit 17 averages the brightness values of the G components that are provided by pixels included in the ROI. Furthermore, instead of averaging, the middle value or the mode value may be calculated, and during the above-described averaging process, arithmetic mean may be executed, or any other averaging operations, such as weighted mean or running mean, may be also executed. Furthermore, the brightness value of the R component or the B component other than the G component may be used, and the brightness values of the wavelength components of RGB may be used. Thus, the brightness value of the G component, representative of the ROI, is obtained for each frame. Then, the calculating unit 17 calculates a difference in the representative value of the ROI between the frame N and the frame N−1. The calculating unit 17 performs calculation, e.g., it subtracts the representative value of the ROI in the frame N−1 from the representative value of the ROI in the frame N, thereby determining the difference in the brightness of the ROI between the frames.

The pulse-wave detecting unit 18 is a processing unit that detects a pulse wave on the basis of a difference in the brightness of the ROI between the frames.

According to an embodiment, the pulse-wave detecting unit 18 sums the difference in the brightness of the ROI, calculated between successive frames. Thus, it is possible to generate pulse wave signals where the amount of change in the brightness of the G component of the ROI is sampled in the sampling period that corresponds to the frame frequency of the image captured by the camera 12. For example, the pulse-wave detecting unit 18 performs the following process each time the calculating unit 17 calculates a difference in the brightness of the ROI. Specifically, the pulse-wave detecting unit 18 adds a difference in the brightness of the ROI between the frame N and the frame N−1 to the sum obtained by summing the difference in the brightness of the ROI between the frames before the image in the frame N is acquired, i.e., the sum obtained by summing the difference in the brightness of the ROI, calculated between the frames from a frame 1 to the frame N−1. Thus, it is possible to generate pulse wave signals up to the sampling time when the Nth frame is acquired. Furthermore, the sum obtained by summing the difference in the brightness of the ROI, calculated between frames in the interval from the frame 1 to the frame that corresponds to each sampling time, is used as the amplitude value of up to the N−1th frame.

Components that deviate from the frequency band that corresponds to human pulse waves may be removed from the pulse wave signals that are obtained as described above. For example, as an example of the removal method, a bandpass filter may be used to extract only the frequency components in the range of a predetermined threshold. As an example of the cutoff frequency of such a bandpass filter, it is possible to set the lower limit frequency that corresponds to 30 bpm, which is the lower limit of the human pulse-wave frequency, and the upper limit frequency that corresponds to 240 bpm, which is the upper limit thereof.

Furthermore, although pulse wave signals are here detected by using the G component in the illustrated case, the brightness value of the R component or the B component other than the G component may be used, or the brightness value of each wavelength component of RGB may be used.

For example, the pulse-wave detecting unit 18 detects pulse wave signals by using time-series data on the representative values of the two wavelength components, i.e., the R component and the G component, which have different light absorption characteristics of blood, among the three wavelength components, i.e., the R component, the G component, and the B component.

A specific explanation is as follows: pulse waves are detected by using more than two types of wavelengths that have different light absorption characteristics of blood, e.g., the G component that has high light absorption characteristics (about 525 nm) and the R component that has low light absorption characteristics (about 700 nm). Heartbeat is in the range from 0.5 Hz to 4 Hz, 30 bpm to 240 bpm in terms of minute; therefore, other components may be regarded as noise components. If it is assumed that noise has no wavelength characteristics or has a little if it does, the components other than 0.5 Hz to 4 Hz in the G signal and the R signal need to be the same; however, due to a difference in the sensitivity of the camera, the level is different. Therefore, if the difference in the sensitivity for the components other than 0.5 Hz to 4 Hz is compensated, and the R component is subtracted from the G component, whereby noise components may be removed and only pulse wave components may be fetched.

For example, the G component and the R component may be represented by using the following Equation (1) and the following Equation (2). In the following Equation (1), “Gs” denotes the pulse wave component of the G signal and “Gn” denotes the noise component of the G signal and, in the following Equation (2), “Rs” denotes the pulse wave component of the R signal and “Rn” denotes the noise component of the R signal. Furthermore, with regard to noise components, there is a difference in the sensitivity between the G component and the R component, and therefore the compensation coefficient k for the difference in the sensitivity is represented by using the following Equation (3).

Ga=Gs+Gn (1)

Ra=Rs+Rn (2)

k=Gn/Rn (3)

If the difference in the sensitivity is compensated and then the R component is subtracted from the G component, the pulse wave component S is obtained by the following Equation (4). If this is changed into the equation that is presented by Gs, Gn, Rs, and Rn by using the above-described Equation (1) and the above-described Equation (2), the following Equation (5) is obtained, and if the above-described Equation (3) is used, k is deleted, and the equation is organized, the following Equation (6) is derived.

S=Ga−kRa (4)

S=Gs+Gn−k(Rs+Rn) (5)

S=Gs−(Gn/Rn)Rs (6)

Here, the G signal and the R signal have different light absorption characteristics of hemoglobin, and Gs>(Gn/Rn)Rs. Therefore, with the above-described Equation (6), it is possible to calculate the pulse wave component S from which noise has been removed.

After the pulse wave signal is obtained as described above, the pulse-wave detecting unit 18 may directly output the waveform of the obtained pulse wave signal as one form of the detection result of the pulse wave, or it may also output the number of pulses that is obtained from the pulse wave signal.

For example, according to an example of the method for calculating the number of pulses, each time the amplitude value of a pulse wave signal is output, detection on the peak of the waveform of the pulse wave signal, e.g., detection on the zero-crossing point of the differentiated waveform, is executed. Here, if the pulse-wave detecting unit 18 detects the peak of the waveform of the pulse wave signal during peak detection, it stores the sampling time when the peak, i.e., the maximum point, is detected in an undepicted internal memory. Then, when the peak appears, the pulse-wave detecting unit 18 obtains the difference in time from the maximum point that is previous by a predetermined parameter n and then divides it by n, thereby detecting the number of pulses. Here, in the illustrated case, the number of pulses is detected by using the peak interval; however, the pulse wave signal is converted into the frequency component so that the number of pulses may be calculated from the frequency that has its peak in the frequency band that corresponds to the pulse wave, e.g., the frequency band of, for example, equal to or more than 40 bpm and equal to or less than 240 bpm.

The number of pulses or the pulse waveform obtained as described above may be output to any output destination, including the display unit 11. For example, if the pulse-wave detection device 10 has a diagnosis program installed therein to diagnose the autonomic nervous function on the basis of fluctuations in the pulse cycle or the number of pulses or to diagnose heart disease, or the like, on the basis of pulse wave signals, the output destination may be the diagnosis program. Furthermore, the output destination may be also the server device, or the like, which provides the diagnosis program as a Web service. Furthermore, the output destination may be also the terminal device that is used by a person related to the user who uses the pulse-wave detection device 10, e.g., a care person or a doctor. This allows monitoring services outside the hospital, e.g., at home or at seat. Furthermore, it is obvious that measurement results or diagnosis results of the diagnosis program may be also displayed on terminal devices of a related person, including the pulse-wave detection device 10.

Furthermore, the acquiring unit 13, the face detecting unit 15, the ROI setting unit 16, the calculating unit 17, and the pulse-wave detecting unit 18, described above, may be implemented when a central processing unit (CPU), a micro processing unit (MPU), or the like, executes the pulse-wave detection program. Furthermore, each of the above-described processing units may be implemented by a hard wired logic, such as an application specific integrated circuit (ASIC) or a field programmable gate array (FPGA).

Furthermore, for example, a semiconductor memory device may be used as the internal memory that is used as a work area by the above-described image storage unit 14 or each processing unit. Examples of the semiconductor memory device include a video random access memory (VRAM), a random access memory (RAM), a read only memory (ROM), or a flash memory. Furthermore, instead of the primary storage device, an external storage device, such as SSD, HDD, or optical disk, may be used.

Furthermore, the pulse-wave detection device 10 may include various functional units included in known computers other than the functional units illustrated in FIG. 1. For example, if the pulse-wave detection device 10 is installed as a stationary terminal, it may further include an input/output device, such a keyboard, a mouse, or a display. Furthermore, if the pulse-wave detection device 10 is installed as a tablet terminal or a slate terminal, it may further include a motion sensor, such as an acceleration sensor or an angular velocity sensor. Moreover, if the pulse-wave detection device 10 is installed as a mobile communication terminal, it may further include a functional unit, such as an antenna, a wireless communication unit connected to a mobile communication network, or a Global Positioning System (GPS) receiver.

Flow of Process

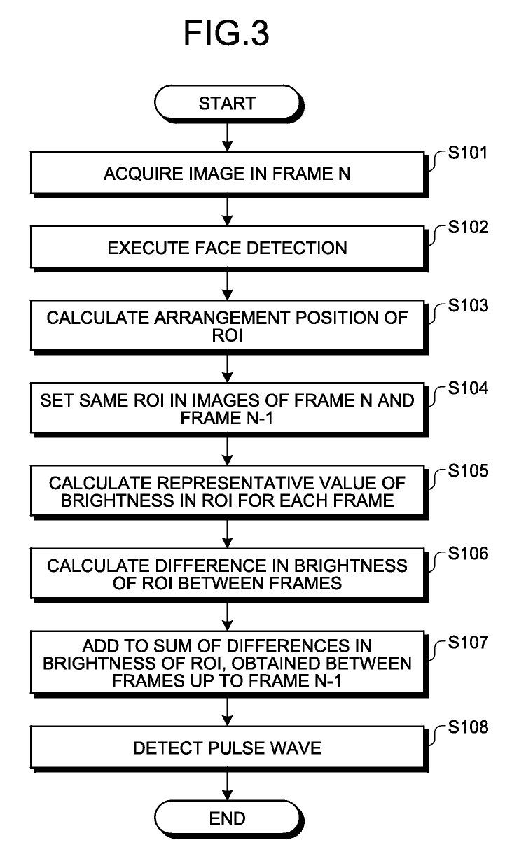

Next, an explanation is given of the flow of a process of the pulse-wave detection device 10 according to the present embodiment. FIG. 3 is a flowchart that illustrates the steps of the pulse-wave detection process according to the first embodiment. This process may be performed if the pulse-wave detection program is active, or it may be also performed if the pulse-wave detection program is operated in the background.

As illustrated in FIG. 3, if the acquiring unit 13 acquires the image in the frame N (Step S101), the face detecting unit 15 executes face detection on the image in the frame N acquired at Step S101 (Step S102).

Next, in accordance with the face detection result of the image in the frame N detected at Step S102, the ROI setting unit 16 calculates the arrangement position of the ROI that is set in the images that correspond to the frame N and the frame N−1 (Step S103). Then, with regard to the two images of the frame N and the frame N−1, the ROI setting unit 16 sets the same ROI in the arrangement position that is calculated at Step S103 (Step S104).

Then, for each of the frame N and the frame N−1, the calculating unit 17 calculates the representative value of the brightness in the ROI that is set in the image of the frame (Step S105). Next, the calculating unit 17 calculates the difference in the brightness of the ROI between the frame N and the frame N−1 (Step S106).

Then, the pulse-wave detecting unit 18 adds the difference in the brightness of the ROI between the frame N and the frame N−1 to the sum obtained by summing the difference in the brightness of the ROI, calculated between the frames from the frame 1 to the frame N−1 (Step S107). Thus, it is possible to obtain the pulse wave signal up to the sampling time in which the Nth frame is acquired.

Then, in accordance with the result of calculation at Step S107, the pulse-wave detecting unit 18 detects the pulse wave signal or the pulse wave, such as the number of pulses, up to the sampling time in which the Nth frame is acquired (Step S108) and terminates the process.

One Aspect of the Advantage

As described above, if the pulse-wave detection device 10 according to the present embodiment sets the ROI to calculate a difference in the brightness from the face detection result of the image captured by the camera 12, it sets the same ROI in the frames and detects a pulse wave signal on the basis of the difference in the brightness within the ROI. Therefore, with the pulse-wave detection device 10 according to the present embodiment, it is possible to prevent a decrease in the accuracy with which pulse waves are detected. Furthermore, with the pulse-wave detection device 10 according to the present embodiment, a lowpass filter is applied to output of the coordinates of the face region so that, without stabilizing changes in the position of the ROI, it is possible to prevent a decrease in the accuracy with which pulse waves are detected. Therefore, it is applicable to real-time processing and, as a result, general versatility may be improved.

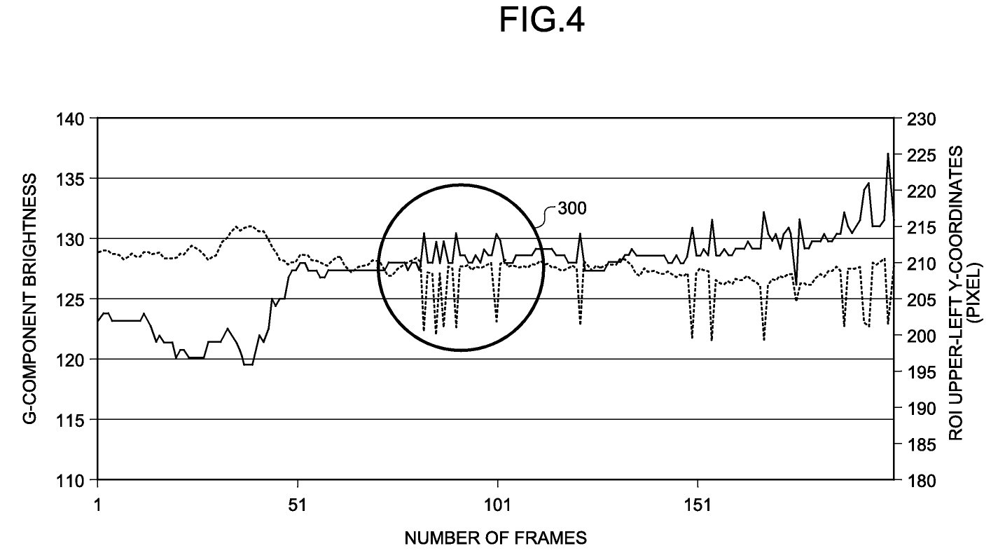

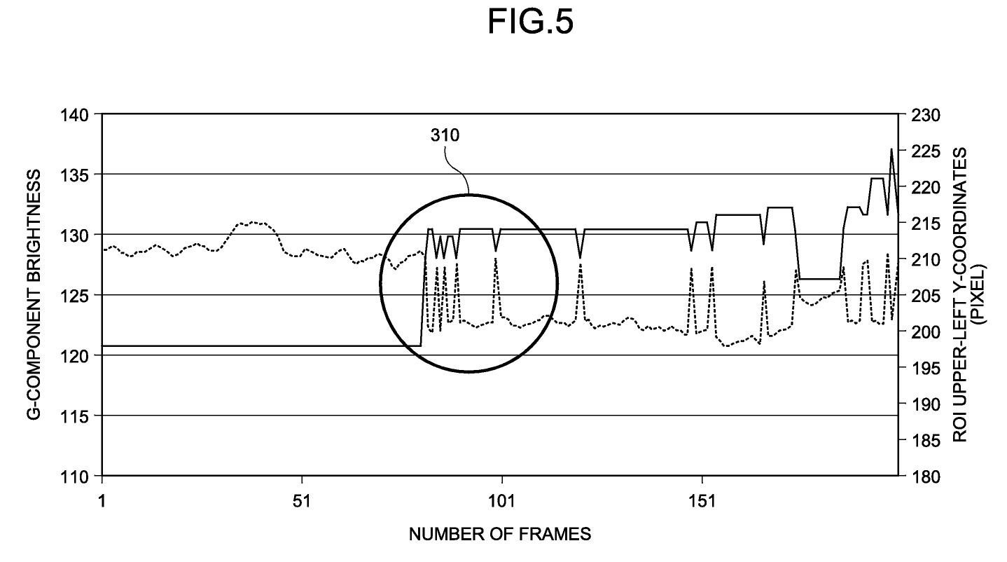

Here, an explanation is given of one aspect of the technical meaning of setting the same ROI in frames. FIGS. 4 and 5 are graphs that illustrate examples of the relationship between a change in the position of the ROI and a change in the brightness. FIG. 4 illustrates a change in the brightness in a case where the ROI is updated in frames in accordance with a face detection result, and FIG. 5 illustrates a change in the brightness in a case where update to the ROI is restricted if the amount of movement of the ROI in frames is equal to or less than a threshold. The dashed line, illustrated in FIGS. 4 and 5, indicates a time change in the brightness value of the G component, and the solid line, illustrated in FIGS. 4 and 5, indicates a time change of the Y-coordinates (in a vertical direction) of the upper left vertex of the rectangle that forms the ROI.

As illustrated in FIG. 4, if update to the ROI in frames is not restricted, it is understood that there occurs noise of equal to or more than the amplitude of a pulse wave signal. For example, in an area 300 of FIG. 4, if the coordinate value of the ROI changes by several pixels, the brightness value of the G component changes by 4 to 5. Generally, as the brightness changes by the amplitude of 1 to 2 due to pulse waves, it is understood that update to the ROI causes noise that is several times as the pulse wave signal.

Conversely, as illustrated in FIG. 5, if update to the ROI in frames is restricted, too, it is understood that there occurs noise of equal to or more than the amplitude of pulse wave signals. Specifically, in an area 310 of FIG. 5, the amount of movement of the ROI exceeds the threshold, and update to the ROI is executed. In this case, as is the case with the area 300 of FIG. 4, the coordinate value of the ROI changes by several pixels, and the brightness value of the G component accordingly changes by 4 to 5.

The above-described noise caused by update to the ROI may be reduced by setting the same ROI in frames as described above. Specifically, by using the knowledge that, in the same ROI within the images of successive frames, a change in the brightness of the pulse is relatively larger than a change in the brightness due to variation in the position of the face, pulse signals with little noise may be detected.

A specific example of the amount of change in both of them in a typical situation is given below.

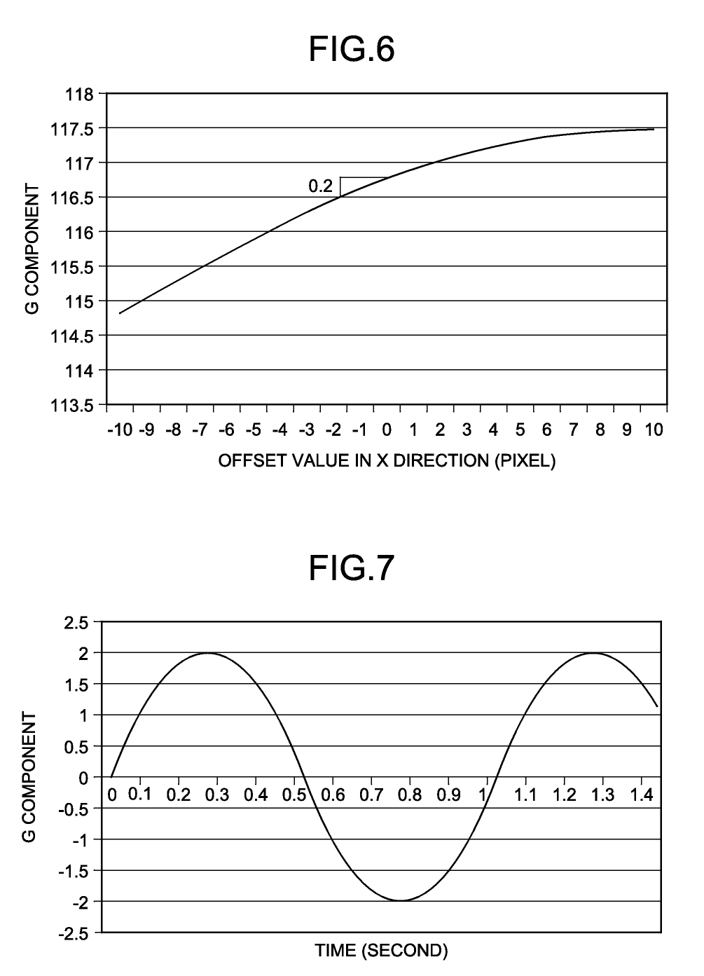

FIG. 6 is a graph that illustrates an example of changes in the brightness due to changes in the position of the face. FIG. 6 illustrates changes in the brightness of the G component if the arrangement position of the ROI, calculated from the face detection result, is moved on the same image in a horizontal direction, i.e., from left to right in the drawing. The vertical axis, illustrated in FIG. 6, indicates the brightness value of the G component, and the horizontal axis indicates the amount of movement, e.g., the offset value, of the X-coordinates (in a horizontal direction) of the upper left vertex of the rectangle that forms the ROI.

As illustrated in FIG. 6, a change in the brightness with the offset of about 0 pixel is about 0.2 per pixel. That is, it can be said that a change in the brightness if the face moves by 1 pixel is “0.2”. Aside from this, if it is assumed that the user moves under the following condition, the amount of movement per frame is about “0.5 pixel” in the actual measurement. Specifically, it assumes the amount of movement of the face if the frame rate of the camera 12 is 20 fps and the resolution of the camera 12 conforms to the standard of Video Graphics Array (VGA). Here, if the user's head moves at the speed of 5 mm/s in a situation where the distance between the camera 12 and the user's face is 30 cm, the user's face moves with the percentage of 0.5 pixel per frame in the actual measurement.

For these reasons, if the user's face moves at the speed of 5 mm/s, the amount of change in the brightness between successive frames is about 0.1 (=0.2×0.5).

Conversely, the amplitude of a change in the brightness due to pulses is about 2. Here, the amount of change is determined when the waveform of a difference in the brightness is represented by using a sine wave if the number of pulses is 60 pulses/minute, i.e., one pulse per second.

FIG. 7 is a graph that illustrates an example of the change in the brightness due to pulses. The vertical axis, illustrated in FIG. 7, indicates a difference in the brightness of the G component, and the horizontal axis, illustrated in FIG. 7, indicates the time (second). As illustrated in FIG. 7, it is understood that, if the frame rate of the camera 12 is 20 fps, the change in the brightness is largest, i.e., about 0.5, at about 0 second to 0.1 second. Therefore, a difference in the brightness of the ROI between successive frames is about 0.5 at a maximum.

As described above, it can be said that a change in the brightness if the position of the face changes with the ROI fixed in successive frames is about 0.1, while a change in the brightness due to pulse changes is about 0.5. Therefore, according to the present embodiment, as the S/N ratio is about 5 and, even if the position of the face changes, it is expected that its effect may be removed to some extent.

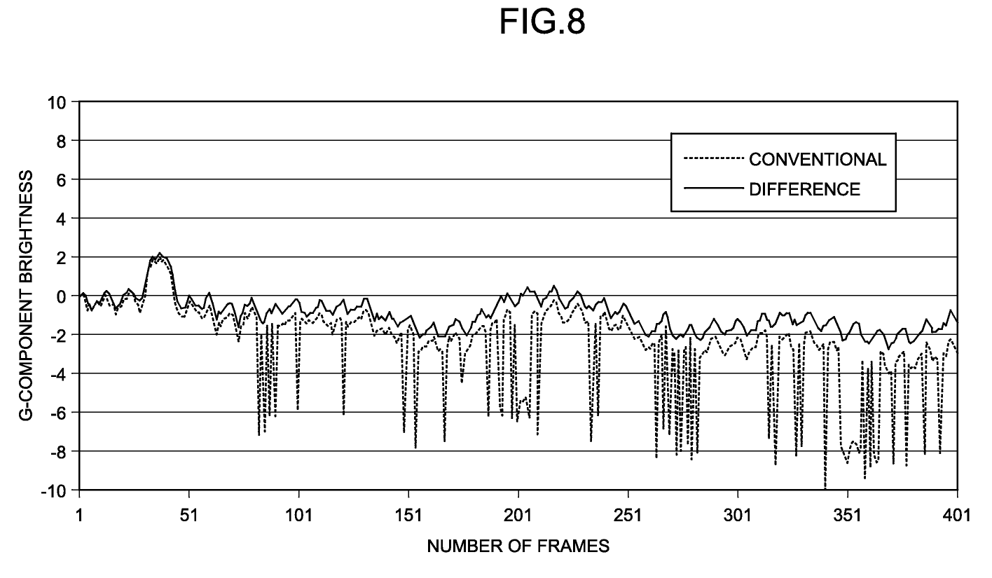

Next, the waveform of a pulse wave signal is illustrated, which is obtained by applying the pulse-wave detection process according to the present embodiment, and it is compared with the pulse wave signal that is obtained in a case where update to the ROI is not restricted. FIG. 8 is a graph that illustrates an example of time changes in the brightness. The vertical axis, illustrated in FIG. 8, indicates a difference in the brightness of the G component, and the horizontal axis, illustrated in FIG. 8, indicates the number of frames. In FIG. 8, the pulse wave signal according to the present embodiment is represented by the solid line, while the pulse wave signal according to a conventional technology, where update to the ROI is not restricted, is represented by the dashed line.

As illustrated in FIG. 8, it is understood that, in the pulse wave signal according to the conventional technology, a change in the brightness is about 5 and there occurs noise that does not appear due to pulses. Conversely, it is understood that the noise, which occurs in the pulse wave signal according to the conventional technology, is reduced in the pulse wave signal according to the present embodiment. Thus, according to the present embodiment, a decrease in the accuracy with which pulse waves are detected may be prevented.

### [b] Second Embodiment

In the case illustrated according to the above-described first embodiment, if a difference in the brightness of the ROI between frames is obtained, the representative value is calculated by uniformly applying the weight for the brightness value of a pixel included in the ROI; however, the weight may be changed for the pixels included in the ROI. Therefore, in the present embodiment, for example, an explanation is given of a case where the representative value of the brightness is calculated by changing the weight for the pixels included in a specific area out of the pixels included in the ROI and for the pixels included in the other areas.

Configuration of a Pulse-Wave Detection Device 20

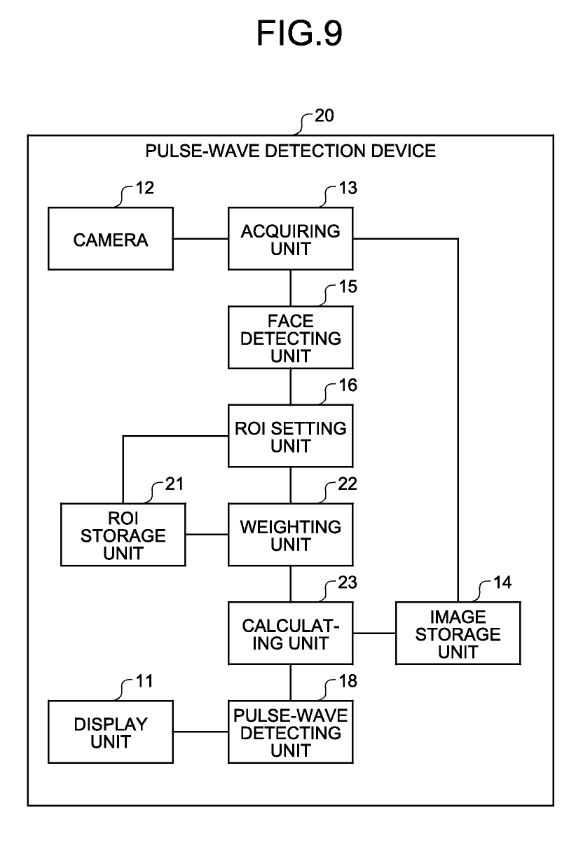

FIG. 9 is a block diagram that illustrates the functional configuration of the pulse-wave detection device 20 according to the second embodiment. The pulse-wave detection device 20 illustrated in FIG. 9 is different from the pulse-wave detection device 10 illustrated in FIG. 1 in that it further includes an ROI storage unit 21 and a weighting unit 22 and part of the processing details of a calculating unit 23 is different from that of the calculating unit 17. Furthermore, the same reference numeral is here applied to the functional unit that performs the same function as that of the functional unit illustrated in FIG. 1, and its explanation is omitted.

The ROI storage unit 21 is a storage unit that stores the arrangement position of the ROI.

According to an embodiment, each time the ROI setting unit 16 sets the ROI, the ROI storage unit 21 registers the arrangement position of the ROI in relation to the frame, of which the image is acquired. For example, when a weight is applied to a pixel included in the ROI, the ROI storage unit 21 refers to the arrangement position of the ROI that is set in the previous or next frame if the frame is previously acquired.

The weighting unit 22 is a processing unit that applies a weight to a pixel included in the ROI.

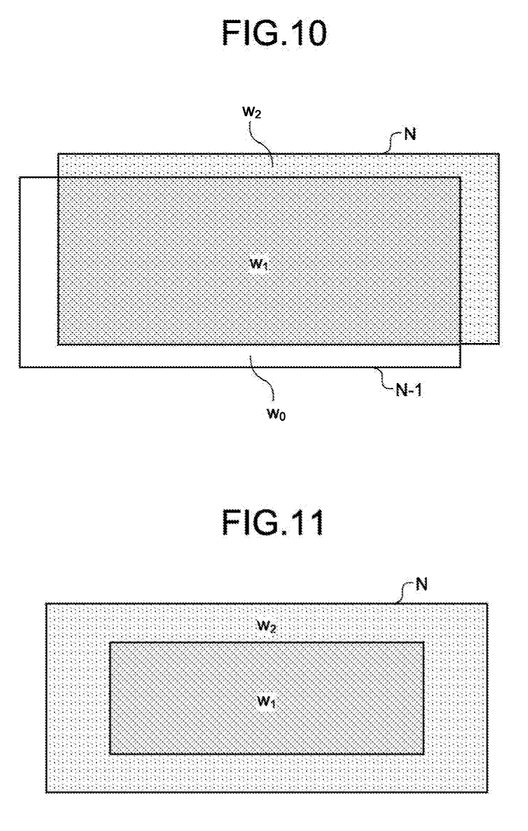

According to an embodiment, the weighting unit 22 applies a low weight to the pixels in the boundary section out of the pixels included in the ROI, compared to the pixels in the other sections. For example, the weighting unit 22 may execute weighting illustrated in FIGS. 10 and 11. FIGS. 10 and 11 are diagrams that illustrate an example of the weighting method. With regard to the painting illustrated in FIGS. 10 and 11, the painting in dark indicates the pixels to which a high weight w₁ is applied as compared to the painting in light, while the painting in light indicates the pixels to which a low weight w₂ is applied as compared to the painting in dark. Here, FIG. 10 illustrates the ROI that is calculated in the frame N−1 together with the ROI that is calculated in the frame N.

For example, in the case of the weighting illustrated in FIG. 10, the weighting unit 22 applies the weight w₁ (>w₂) to the section where the ROI in the frame N−1 and the ROI in the frame N are overlapped with each other, that is, the pixels included in the painting in dark, out of the ROI that is calculated by the ROI setting unit 16 when the frame N is acquired. Furthermore, the weighting unit 22 applies the weight w₂ (<w₁) to the section where the ROI in the frame N−1 and the ROI in the frame N are not overlapped with each other, that is, the pixels included in the painting in light. Thus, it is possible that the weight for the section where the ROIs in frames are overlapped is higher than that for the section where they are not overlapped and, as a result, there is a higher possibility that a change in the brightness used for summation may be obtained from the same region on the face.

Furthermore, in the case of the weighting illustrated in FIG. 11, the weighting unit 22 applies the weight w₂ (<w₁) to the area within a predetermined range from each of the sides that form the ROI, that is, the pixels included in the area painted in light, out of the ROI that is calculated by the ROI setting unit 16 when the frame N is acquired. Furthermore, the weighting unit 22 applies the weight w₁ (>w₂) to the area out of the predetermined range from each of the sides that form the ROI, that is, the pixels included in the area painted in dark, out of the ROI that is calculated by the ROI setting unit 16 when the frame N is acquired. Thus, it is possible that the weight for the boundary section of the ROI is lower than that for the central section and, as a result, there is a higher possibility that a change in the brightness used for summation may be obtained from the same region on the face, as is the case with the example of FIG. 9.

The calculating unit 23 performs an operation on each frame to do the weighted mean of the pixel value of each pixel in the ROI in accordance with the weight w₁ and the weight w₂ that are applied to the pixels in the ROIs in the frame N and the frame N−1, respectively, by the weighting unit 22. Thus, the representative value of the brightness in the ROI of the frame N and the representative value of the brightness in the ROI of the frame N−1 are calculated. With regard to the other operations, the calculating unit 23 performs the same operation as that of the calculating unit 17 illustrated in FIG. 1.

Flow of Process

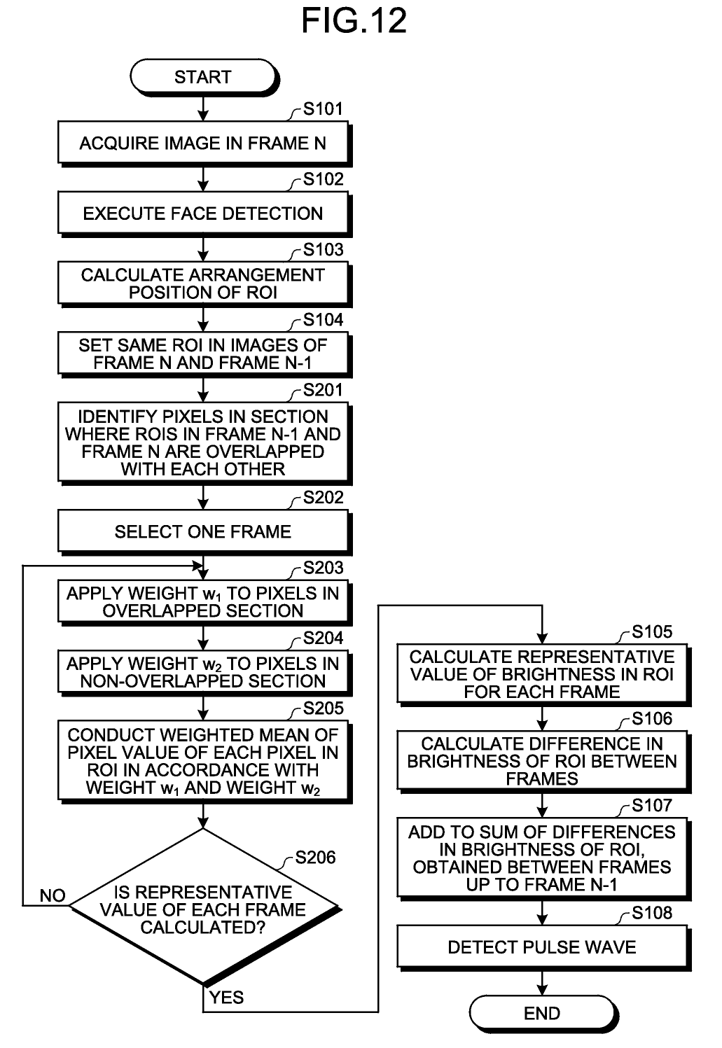

FIG. 12 is a flowchart that illustrates the steps of the pulse-wave detection process according to the second embodiment. In the same manner as the case illustrated in FIG. 3, this process may be performed if the pulse-wave detection program is active, or it may be also performed if the pulse-wave detection program is operated in the background. Here, FIG. 12 illustrates the flowchart in a case where, among the weighting methods, the weighting illustrated in FIG. 10 is applied, and the different reference numerals are applied to the parts of which the processing details are different from those in the flowchart illustrated in FIG. 3.

As illustrated in FIG. 12, if the acquiring unit 13 acquires the image in the frame N (Step S101), the face detecting unit 15 executes face detection on the image in the frame N acquired at Step S101 (Step S102).

Next, in accordance with the face detection result of the image in the frame N detected at Step S102, the ROI setting unit 16 calculates the arrangement position of the ROI that is set in the images that correspond to the frame N and the frame N−1 (Step S103). Then, with regard to the two images in the frame N and the frame N−1, the ROI setting unit 16 sets the same ROI in the arrangement position that is calculated at Step S103 (Step S104).

Then, in the ROI that is calculated at Step S103, the weighting unit 22 identifies the pixels in the section where the ROI in the frame N−1 and the ROI in the frame N are overlapped with each other (Step S201).

Then, the weighting unit 22 selects one frame from the frame N−1 and the frame N (Step S202). Then, the weighting unit 22 applies the weight w₁ (>w₂) to the pixels that are determined to be in the overlapped section at Step S201 among the pixels included in the ROI of the frame that is selected at Step S202 (Step S203). Furthermore, the weighting unit 22 applies the weight w₂ (<w₁) to the pixels in the non-overlapped section, which is not determined to be the overlapped section at Step S201, among the pixels included in the ROI of the frame that is selected at Step S202 (Step S204).

Then, the calculating unit 23 executes the weighted mean of the brightness value of each pixel included in the ROI of the frame selected at Step S202 in accordance with the weight w₁ and the weight w₂ that are applied at Steps S203 and S204 (Step S205). Thus, the representative value of the brightness in the ROI of the frame selected at Step S202 is calculated.

Then, the above-described process from Step S203 to Step S205 is repeatedly performed until the representative value of the brightness in the ROI of each of the frame N−1 and the frame N is calculated (No at Step S206).

Then, if the representative value of the brightness in the ROI of each of the frame N−1 and the frame N is calculated (Yes at Step S206), the calculating unit 23 performs the following operation. That is, the calculating unit 23 calculates a difference in the brightness of the ROI between the frame N and the frame N−1 (Step S106).

Then, the pulse-wave detecting unit 18 adds the difference in the brightness of the ROI between the frame N and the frame N−1 to the sum obtained by summing the difference in the brightness of the ROI, calculated between the frames from the frame 1 to the frame N−1 (Step S107). Thus, it is possible to obtain the pulse wave signal up to the sampling time in which the Nth frame is acquired.

Then, in accordance with the result of calculation at Step S107, the pulse-wave detecting unit 18 detects the pulse wave signal or the pulse wave, such as the number of pulses, up to the sampling time in which the Nth frame is acquired (Step S108) and terminates the process.

One Aspect of the Advantage

As described above, if the pulse-wave detection device 20 according to the present embodiment also sets the ROI to calculate a difference in the brightness from the face detection result of the image captured by the camera 12, it sets the same ROI in the frames and detects a pulse wave signal on the basis of the difference in the brightness within the ROI. Therefore, with the pulse-wave detection device 20 according to the present embodiment, it is possible to prevent a decrease in the accuracy with which pulse waves are detected in the same manner as the above-described first embodiment.

Furthermore, with the pulse-wave detection device 20 according to the present embodiment, the weight for the section where the ROIs in frames are overlapped may be higher than that for the non-overlapped section and, as a result, there is a higher possibility that a change in the brightness used for summation may be obtained from the same region on the face.

### [c] Third Embodiment

In the case illustrated according to the above-described first embodiment, if a difference in the brightness of the ROI between frames is obtained, the representative value is calculated by uniformly applying the weight for the brightness value of a pixel included in the ROI; however, all the pixels included in the ROI do not need to be used for calculation of the representative value of the brightness. Therefore, in the present embodiment, an explanation is given of a case where, for example, the ROI is divided into blocks and blocks, which satisfy a predetermined condition among the blocks, are used for calculation of the representative value of the brightness in the ROI.

Configuration of a Pulse-Wave Detection Device 30

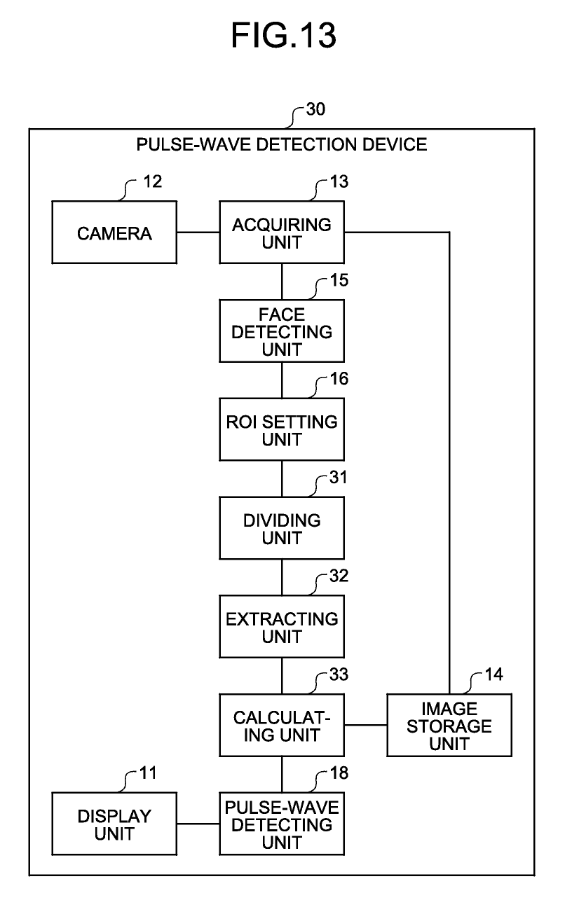

FIG. 13 is a block diagram that illustrates a functional configuration of the pulse-wave detection device 30 according to a third embodiment. The pulse-wave detection device 30 illustrated in FIG. 13 is different from the pulse-wave detection device 10 illustrated in FIG. 1 in that it further includes a dividing unit 31 and an extracting unit 32 and part of the processing details of a calculating unit 33 is different from that of the calculating unit 17. Furthermore, the same reference numeral is here applied to the functional unit that performs the same function as that of the functional unit illustrated in FIG. 1, and its explanation is omitted.

The dividing unit 31 is a processing unit that divides the ROI.

According to an embodiment, the dividing unit 31 divides the ROI, set by the ROI setting unit 16, into a predetermined number of blocks, e.g., 6×9 blocks in vertical and horizontal. In the case illustrated here, the ROI is divided into blocks; however, it does not always need to be divided in a block shape, but it may be divided in any other shapes.

The extracting unit 32 is a processing unit that extracts a block that satisfies a predetermined condition among the blocks that are divided by the dividing unit 31.

According to an embodiment, the extracting unit 32 selects one block from the blocks that are divided by the dividing unit 31. Next, with regard to each of the blocks located in the same position in the frame N and the frame N−1, the extracting unit 32 calculates a difference in the representative value of the brightness between the blocks. Then, if a difference in the representative value of the brightness between the blocks located in the same position on the image is less than a predetermined threshold, the extracting unit 32 extracts the block as the target for calculation of a change in the brightness. Then, the extracting unit 32 repeatedly performs the above-described threshold determination until all the blocks, divided by the dividing unit 31, are selected.

The calculating unit 33 uses the brightness value of each pixel in the block, extracted by the extracting unit 32, among the blocks divided by the dividing unit 31 to calculate the representative value of the brightness in the ROI for each of the frame N and the frame N−1. Thus, the representative value of the brightness in the ROI of the frame N and the representative value of the brightness in the ROI of the frame N−1 are calculated. As for the other processes, the calculating unit 33 performs the same process as that of the calculating unit 17 illustrated in FIG. 1.

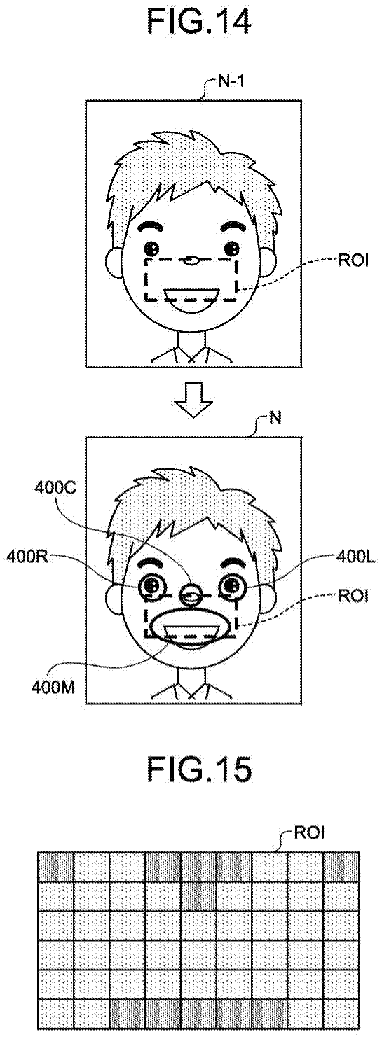

FIG. 14 is a diagram that illustrates an example of shift of the ROI. FIG. 15 is a diagram that illustrates an example of extraction of a block. As illustrated in FIG. 14, if the arrangement position of the ROI calculated from the frame N is shifted upward in a vertical direction from the arrangement position of the ROI calculated from the frame N−1, there occurs a deviation in the region of which a change in the brightness is calculated, and the ROI includes the region where its brightness gradient is high on the face. That is, the ROI includes part of a left eye 400L, a right eye 400R, a nose 400C, and a mouth 400M. Although these facial parts with a high brightness gradient cause noise, the block that includes some of the facial part, such as the left eye 400L, the right eye 400R, the nose 400C, or the mouth 400M may be eliminated from the target for calculation of the representative value of the brightness in the ROI due to the threshold determination by the extracting unit 32, as illustrated in FIG. 15. As a result, it is possible to prevent a situation where changes in the brightness of a facial part, included in the ROI, are larger than pulses.

Furthermore, if the percentage of blocks, of which a difference in the representative value of the brightness between the blocks located in the same position is equal to or more than a threshold, is a predetermined percentage, e.g., more than two thirds, or if the amount of positional movement from the ROI in the frame N−1 is large, there is a high possibility that the arrangement position of the ROI in the current frame N is not reliable; therefore, the arrangement position of the ROI calculated in the frame N−1 may be used instead of the arrangement position of the ROI calculated in the frame N. Furthermore, if the amount of movement from the ROI in the frame N−1 is small, the process may be canceled.

Flow of Process

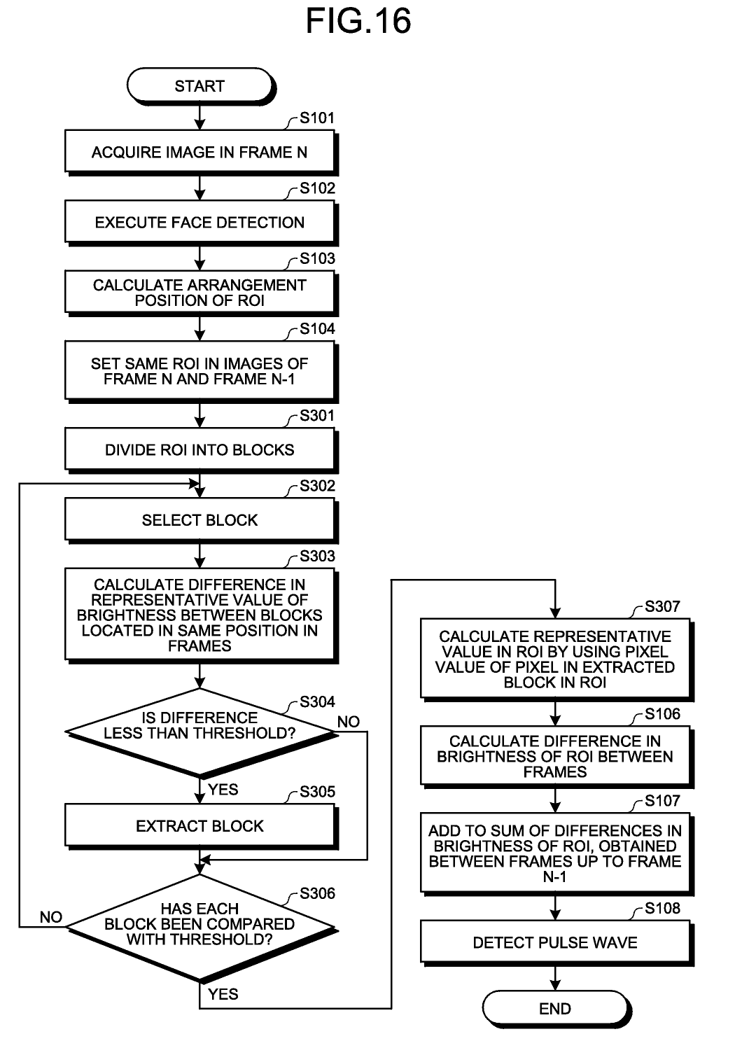

FIG. 16 is a flowchart that illustrates the steps of a pulse-wave detection process according to the third embodiment. In the same manner as the case illustrated in FIG. 3, this process may be performed if the pulse-wave detection program is active, or it may be also performed if the pulse-wave detection program is operated in the background. Here, in FIG. 13, the different reference numerals are applied to the parts of which the processing details are different from those in the flowchart illustrated in FIG. 3.

As illustrated in FIG. 16, if the acquiring unit 13 acquires the image in the frame N (Step S101), the face detecting unit 15 executes face detection on the image in the frame N acquired at Step S101 (Step S102).

Next, in accordance with the face detection result of the image in the frame N detected at Step S102, the ROI setting unit 16 calculates the arrangement position of the ROI that is set in the images that correspond to the frame N and the frame N−1 (Step S103). Then, with regard to the two images in the frame N and the frame N−1, the ROI setting unit 16 sets the same ROI in the arrangement position that is calculated at Step S103 (Step S104).

Then, the dividing unit 31 divides the ROI, set at Step S104, into blocks (Step S301). Next, the extracting unit 32 selects one block from the blocks that are divided at Step S301 (Step S302).

Then, for each of the blocks located in the same position in the frame N and the frame N−1, the extracting unit 32 calculates a difference in the representative value of the brightness between the blocks (Step S303). Then, the extracting unit 32 determines whether a difference in the representative value of the brightness between the blocks located in the same position on the image is less than a predetermined threshold (Step S304).

Here, if a difference in the representative value of the brightness between the blocks located in the same position on the image is less than the threshold (Yes at Step S304), it may be assumed that there is a high possibility that the block does not include a facial part, or the like, which has a high brightness gradient. In this case, the extracting unit 32 extracts the block as the target for calculation of a change in the brightness (Step S305). Conversely, if a difference in the representative value of the brightness between the blocks located in the same position on the image is equal to or more than the threshold (No at Step S304), it may be assumed that there is a high possibility that the block includes a facial part, or the like, which has a high brightness gradient. In this case, the block is not extracted as the target for calculation of a change in the brightness, and a transition is made to Step S306.

Then, the extracting unit 32 repeatedly performs the above-described process from Step S302 to Step S305 until each of the blocks, divided at Step S301, is selected (No at Step S306).

Then, after each of the blocks, divided at Step S301, is selected (Yes at Step S306), the representative value of the brightness in the ROI is calculated for each of the frame N and the frame N−1 by using the brightness value of each pixel in the block extracted at Step S305 among the blocks divided at Step S301 (Step S307). Next, the calculating unit 23 calculates a difference in the brightness of the ROI between the frame N and the frame N−1 (Step S106).

Then, the pulse-wave detecting unit 18 adds the difference in the brightness of the ROI between the frame N and the frame N−1 to the sum obtained by summing the difference in the brightness of the ROI, calculated between the frames from the frame 1 to the frame N−1 (Step S107). Thus, it is possible to obtain the pulse wave signal up to the sampling time in which the Nth frame is acquired.

Then, in accordance with the result of calculation at Step S107, the pulse-wave detecting unit 18 detects the pulse wave signal or the pulse wave, such as the number of pulses, up to the sampling time in which the Nth frame is acquired (Step S108) and terminates the process.

One Aspect of the Advantage

As described above, if the pulse-wave detection device 30 according to the present embodiment also sets the ROI to calculate a difference in the brightness from the face detection result of the image captured by the camera 12, it sets the same ROI in the frames and detects a pulse wave signal on the basis of the difference in the brightness within the ROI. Therefore, with the pulse-wave detection device 30 according to the present embodiment, it is possible to prevent a decrease in the accuracy with which pulse waves are detected in the same manner as the above-described first embodiment.

Furthermore, with the pulse-wave detection device 30 according to the present embodiment, the ROI is divided into blocks and, if a difference in the representative value of the brightness between the blocks located in the same position is less than a predetermined threshold, the block is extracted as the target for calculation of a change in the brightness. Therefore, with the pulse-wave detection device 30 according to the present embodiment, the block that includes some of a facial part may be eliminated from the target for calculation of the representative value of the brightness in the ROI and, as a result, it is possible to prevent a situation where changes in the brightness of a facial part, included in the ROI, are larger than pulses.

### [d] Fourth Embodiment

Furthermore, although the embodiments of the disclosed device are described above, the present invention may be implemented in various different embodiments other than the above-described embodiments. Therefore, an explanation is given below of other embodiments included in the present invention.

### APPLICATION EXAMPLES

In the cases illustrated according to the above-described first embodiment to third embodiment, the size of the ROI is fixed; however, the size of the ROI may be changed each time a change in the brightness is calculated. For example, if the amount of movement of the ROI between the frame N and the frame N−1 is equal to or more than a predetermined threshold, the ROI in the frame N−1 may be narrowed down to the section with the weight w₁, which is described in the above-described second embodiment.

### Other Implementation Examples

In the cases illustrated in the above-described first embodiment to third embodiment, the pulse-wave detection devices 10 to 30 perform the above-described pulse-wave detection process on stand-alone; however, they may be implemented as a client server system. For example, the pulse-wave detection devices 10 to 30 may be implemented as a Web server that executes the pulse-wave detection process, or they may be implemented as a cloud that provides the service implemented during the pulse-wave detection process through outsourcing. As described above, if the pulse-wave detection devices 10 to 30 are operated as server devices, mobile terminal devices, such as smartphones or mobile phones, or information processing devices, such as personal computers, may be included as client terminals. If an image is acquired from the client terminal via a network, the above-described pulse-wave detection process is performed, and the detection result of pulse waves or the diagnosis result obtained by using the detection result are replied to the client terminal, whereby a pulse-wave detection service may be provided.

Pulse-Wave Detection Program

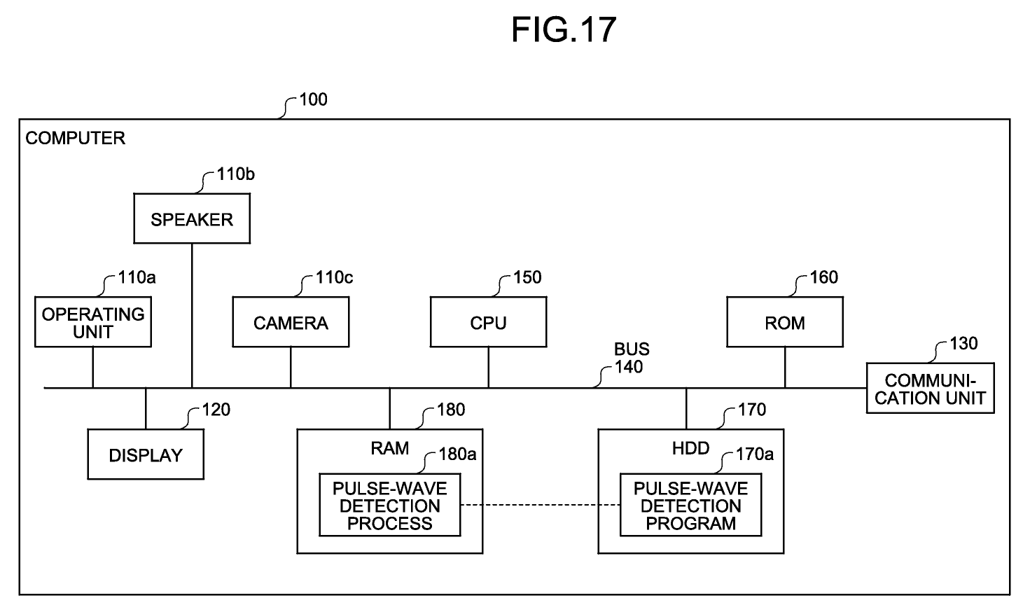

Furthermore, various processes, described in the above-described embodiments, may be performed when a computer, such as a personal computer or a workstation, executes a prepared program. Therefore, with reference to FIG. 17, an explanation is given below of an example of the computer that executes the pulse-wave detection program that has the same functionality as those in the above-described embodiments.

FIG. 17 is a diagram that illustrates an example of the computer that executes the pulse-wave detection program according to the first embodiment to the fourth embodiment. As illustrated in FIG. 17, a computer 100 includes an operating unit 110a, a speaker 110b, a camera 110c, a display 120, and a communication unit 130. The computer 100 includes a CPU 150, a ROM 160, an HDD 170, and a RAM 180. The operating unit 110a, the speaker 110b, the camera 110c, the display 120, the communication unit 130, the CPU 150, the ROM 160, the HDD 170, and the RAM 180 are connected to one another via a bus 140.

As illustrated in FIG. 17, the HDD 170 stores a pulse-wave detection program 170a that performs the same functionality as those of each processing unit illustrated according to the above-described first embodiment to third embodiment. With regard to the pulse-wave detection program 170a, too, integration or separation may be executed in the same manner as each of the processing units illustrated in FIGS. 1, 9, and 13. Specifically, with regard to the data stored in the HDD 170, the entire data does not need to be always stored in the HDD 170, and data used for a process may be stored in the HDD 170.

Furthermore, the CPU 150 reads the pulse-wave detection program 170a from the HDD 170 and loads it into the RAM 180. Thus, as illustrated in FIG. 17, the pulse-wave detection program 170a functions as a pulse-wave detection process 180a. The pulse-wave detection process 180a loads various types of data, read from the HDD 170, into the area assigned thereto in the RAM 180, and it performs various processes on the basis of various types of data loaded. The pulse-wave detection process 180a includes the process performed by each of the processing units illustrated in FIGS. 1, 9, and 13, e.g., the processes illustrated in FIGS. 3, 12, and 16. Furthermore, with regard to the processing units, which are virtually implemented in the CPU 150, all the processing units do not always need to be operated in the CPU 150, and the processing unit used for a process may be virtually operated.

Furthermore, the above-described pulse-wave detection program 170a does not always need to be initially stored in the HDD 170 or the ROM 160. For example, each program is stored in a “portable physical medium”, such as a flexible disk, what is called FD, CD-ROM, DVD disk, magnetic optical disk, or IC card, which is inserted into the computer 100. Furthermore, the computer 100 may acquire each program from the portable physical medium and execute it. Furthermore, a different computer or a server device, connected to the computer 100 via a public network, the Internet, a LAN, a WAN, or the like, may store each program so that the computer 100 acquires each program from them and executes it.

It is possible to prevent a decrease in the accuracy with which pulse waves are detected.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A pulse-wave detection method comprising:
acquiring, by a processor, an image;
executing, by the processor, face detection on the image;
setting, by the processor, an identical region of interest in a frame, of which the image is acquired, and a previous frame to the frame in accordance with a result of the face detection; and
detecting, by the processor, a pulse wave signal based on a difference in brightness obtained between the frame and the previous frame.

2. The pulse-wave detection method according to claim 1, further comprising:
when the region of interest is set, applying, by the processor, a high weight to a pixel in a section where the region of interest is overlapped with a region of interest that is set before the region of interest is set, as compared to a pixel in a non-overlapped section; and
performing, by the processor, an averaging process on a brightness value of each pixel in the region of interest in accordance with the weight that is applied to each pixel in the region of interest for each of the frame and the previous frame.

3. The pulse-wave detection method according to claim 1, further comprising:
when the region of interest is set, applying, by the processor, different weights to a pixel that is present in a boundary section that is in an outer circumference that forms the region of interest and to a pixel that is present in a center section that forms the region of interest; and
performing, by the processor, an averaging process on a brightness value of each pixel in the region of interest in accordance with the weight that is applied to each pixel in the region of interest for each of the frame and the previous frame.

4. The pulse-wave detection method according to claim 1, further comprising:
dividing, by the processor, the region of interest into blocks;
when a difference in a representative value of brightness between blocks located in an identical position in the frame and the previous frame is less than a predetermined threshold, extracting the block, by the processor; and
calculating, by the processor, a representative value of brightness in the region of interest by using a brightness value of each pixel in the extracted block for each of the frame and the previous frame.

5. A non-transitory computer-readable recording medium having stored therein a program that causes a computer to execute a process comprising:
acquiring an image;
executing face detection on the image;
setting an identical region of interest in a frame, of which the image is acquired, and a previous frame to the frame in accordance with a result of the face detection; and
detecting a pulse wave signal based on a difference in brightness obtained between the frame and the previous frame.

6. The computer-readable recording medium according to claim 5, the process further comprising:
when the region of interest is set, applying a high weight to a pixel in a section where the region of interest is overlapped with a region of interest that is set before the region of interest is set, as compared to a pixel in a non-overlapped section; and
performing an averaging process on a brightness value of each pixel in the region of interest in accordance with the weight that is applied to each pixel in the region of interest for each of the frame and the previous frame.

7. The computer-readable recording medium according to claim 5, the process further comprising:
when the region of interest is set, applying different weights to a pixel that is present in a boundary section that is in an outer circumference that forms the region of interest and to a pixel that is present in a center section that forms the region of interest; and
performing an averaging process on a brightness value of each pixel in the region of interest in accordance with the weight that is applied to each pixel in the region of interest for each of the frame and the previous frame.

8. The computer-readable recording medium according to claim 5, the process further comprising:
dividing the region of interest into blocks;
when a difference in a representative value of brightness between blocks located in an identical position in the frame and the previous frame is less than a predetermined threshold, extracting the block; and
calculating a representative value of brightness in the region of interest by using a brightness value of each pixel in the extracted block for each of the frame and the previous frame.

9. A pulse-wave detection device comprising:
a processor configured to;
acquire an image;
execute face detection on the image;
set an identical region of interest in a frame, of which the image is acquired, and a previous frame to the frame in accordance with a result of the face detection; and
detect a pulse wave signal based on a difference in brightness obtained between the frame and the previous frame.

10. The pulse-wave detection device according to claim 9, wherein the processor is configured to:
when the region of interest is set, apply a high weight to a pixel in a section where the region of interest is overlapped with a region of interest that is set before the region of interest is set, as compared to a pixel in a non-overlapped section; and
perform an averaging process on a brightness value of each pixel in the region of interest in accordance with the weight that is applied to each pixel in the region of interest for each of the frame and the previous frame.

11. The pulse-wave detection device according to claim 9, wherein the processor is configured to:
when the region of interest is set, apply different weights to a pixel that is present in a boundary section that is in an outer circumference that forms the region of interest and to a pixel that is present in a center section that forms the region of interest; and
perform an averaging process on a brightness value of each pixel in the region of interest in accordance with the weight that is applied to each pixel in the region of interest for each of the frame and the previous frame.

12. The pulse-wave detection device according to claim 9, wherein the processor is configured to:
divide the region of interest into blocks;
when a difference in a representative value of brightness between blocks located in an identical position in the frame and the previous frame is less than a predetermined threshold, extract the block; and
calculate a representative value of brightness in the region of interest by using a brightness value of each pixel in the extracted block for each of the frame and the previous frame.
